(19) United States
(12) Patent Application Publication
(10) Pub. No.: US 2026/0096745 A1
(43) Pub. Date: Apr. 9, 2026

(54) CEPHALOMETRIC PARAMETERS FOR ASSESSING THE HARMONY, AESTHETICS AND PROPORTIONS OF THE FACE

(71) Applicant: Ksenia AVDOSHENKO, Moscow (RU)

(72) Inventors: Ksenia AVDOSHENKO, Moscow (RU); Nadezhda STRELKOVA, Moscow (RU)

(21) Appl. No.: 19/339,402

(22) Filed: Sep. 25, 2025

Related U.S. Application Data
(60) Provisional application No. 63/702,690, filed on Oct. 3, 2024.

(51) Int. Cl. A61B 5/107; A61B 5/00

(52) U.S. Cl. CPC A61B 5/1079; A61B 5/7271; A61B 5/74

(57) ABSTRACT

A method and apparatus for performing a photometric cephalometric analysis are provided. An image of a face of a patient in a frontal plane is generated such that his/her head is along the Frankfurt horizontal plane. A facial proportionality angle (FPA) is calculated based on the image, with the FPA being formed by two rays going from a vertex located at a gnathion point to zygomatic points on both face sides. The gnathion point is a point of maximum projection of a soft-tissue part of the patient's chin in a sagittal plane. The zygomatic points aligned with each other in a transverse plane are on a skin surface above the most protruding points of zygomatic bones. The FPA is used to determine a relationship between a visual interzygomatic distance (distance between the zygomatic points) and a visual morphological face height (distance between the gnathion point and a soft-tissue nasion point).

### TECHNICAL FIELD

The present disclosure relates to the field of medical imaging and diagnostic analysis. In particular, the present disclosure relates to a method and apparatus for performing a photometric cephalometric analysis based on facial images of patients.

### BACKGROUND OF THE INVENTION

The aesthetics of the face are characterized by proportionality, symmetry, and the interdependence of the sizes of its individual parts, which together create a harmonious image. There are many anthropometric methods to determine facial proportions, which include measuring the ratio of different parts of the head and skull. The most used in aesthetic medicine is the morphological index of the face according to Garson, proposed back in 1887, which is a facial index that allows one to assess the ratio of the morphological width of the face (i.e., the width of the face in the area of the zygomatic arches) and the morphological height of the face (i.e., the height of the face from the nasal suture to the lower chin point).

Today, however, there is a discrepancy in craniofacial identification, as it is common to find descriptions of the facial morphologic index, such as that of Farkas, with the difference being measured by skin points rather than craniometric points. Even though there are several recognized nomenclatures of anthropometric indicators, there is no single agreement in measuring the facial morphologic index. Furthermore, there is a discrepancy between the facial morphologic index according to Garson and the visual assessment of the face due to the fact that the facial morphologic index initially did not involve measuring the soft tissue cover of the human head, the volume of which can be different depending on the age, gender and race of the subjects, as well as did not take into account age-related changes and maxillofacial anomalies affecting the height and width of the face.

### SUMMARY OF THE INVENTION

This summary is provided to introduce a selection of concepts in a simplified form that are further described below in the detailed description. This summary is not intended to identify key features of the present disclosure, nor is it intended to be used to limit the scope of the present disclosure.

It is an objective of the present disclosure to provide new cephalometric parameters for assessing facial harmony, aesthetics, and proportions that account for true visual height and width of the face, eliminate errors in visual assessment of the face, and are simple to apply in clinical practice.

The objective above is achieved by the features of the independent claims in the appended claims. Further embodiments and examples are apparent from the dependent claims, the detailed description, and the accompanying drawings.

According to a first aspect, a method for performing a photometric cephalometric analysis is provided. The method starts with the step of generating an image of a face of a patient in a frontal plane such that his/her head is positioned along a Frankfurt horizontal plane. Then, the method proceeds to the step of using the image to calculate a facial proportionality angle. The facial proportionality angle is formed by two rays going from a vertex located at a gnathion point to zygomatic points on both sides of the face. The gnathion point is a point of maximum projection of a soft-tissue part of a chin of the patient in a sagittal plane, and the zygomatic points are aligned with each other in a transverse plane and located on a skin surface above most protruding (maximum) points of zygomatic bones on both sides of the face. After that, the method goes on to the step of using the facial proportionality angle to determine a relationship between a visual interzygomatic distance and a visual morphological height of the face. The visual interzygomatic distance is a distance between the zygomatic points, and the visual morphological height of the face is a distance between the gnathion point and a soft-tissue nasion point.

By using the method according to the first aspect, the aesthetic parameters of patients' faces may be studied and evaluated more efficiently, safely and easily, which is important for personalized planning and improving the quality of aesthetic interventions and increasing patient satisfaction. The FPA so defined allows one to more accurately assess the visual proportions of the face and reasonably approach the planning of rejuvenating operations and procedures, as well as evaluate the result of facial harmonization. Another undeniable advantage of the method according to the first aspect is that it is easy to apply using a full-face photo of the patient, regardless of its scale—this greatly facilitates photo counseling and online planning for non-resident patients.

In one exemplary embodiment of the first aspect, if the facial proportionality angle is less than 70 degrees, the visual morphological height of the face is determined to prevail over the visual interzygomatic distance, meaning that the face of the patient is long and narrow. At the same time, if the facial proportionality angle is more than 75 degrees, the visual interzygomatic distance is determined to prevail over the visual morphological height of the face, meaning that the face of the patient is short and wide.

According to a second aspect, an apparatus for performing a photometric cephalometric analysis is provided, which comprises at least one processor and at least one memory coupled to the at least one processor and storing processor-executable instructions. Being executed by the at least one processor, the processor-executable instructions cause the at least one processor to operate as follows. At first, the at least one processor receives an image of a face of a patient in a frontal plane, with the image showing a head of the patient positioned along a Frankfurt horizontal plane. Then, the at least one processor uses the image to calculate a facial proportionality angle. The facial proportionality angle is formed by two rays going from a vertex located at a gnathion point to zygomatic points on both sides of the face. The gnathion point is a point of maximum projection of a soft-tissue part of a chin of the patient in a sagittal plane, and the zygomatic points are aligned with each other in a transverse plane and located on a skin surface above most protruding points of zygomatic bones on both sides of the face. Next, the at least one processor uses the facial proportionality angle to determine a relationship between a visual interzygomatic distance and a visual morphological height of the face. The visual interzygomatic distance is a distance between the zygomatic points, and the visual morphological height of the face is a distance between the gnathion point and a soft-tissue nasion point. After that, the at least one processors can output, to a user (e.g., physician), the determined relationship between the visual interzygomatic distance and the visual morphological height of the face.

The apparatus according to the second aspect provides the same or similar advantages as those discussed above with respect to the method according to the first aspect.

In one exemplary embodiment of the second aspect, if the facial proportionality angle is less than 70 degrees, the at least one processor determines that the visual morphological height of the face prevails over the visual interzygomatic distance, meaning that the face of the patient is long and narrow. At the same time, if the facial proportionality angle is more than 75 degrees, the at least one processor determines that the visual interzygomatic distance prevails over the visual morphological height of the face, meaning that the face of the patient is short and wide.

Other features and advantages of the present disclosure will be apparent upon reading the following detailed description and reviewing the accompanying drawings.

### BRIEF DESCRIPTION OF DRAWINGS

The present disclosure is explained below with reference to the accompanying drawings in which:

FIGS. 1A and 1B show the location of craniometric points on the skull (FIG. 1A) and relative to the soft tissue cover of the head (FIG. 1B), which are used to calculate the facial morphologic index according to Garson;

FIGS. 2A-2C show faces of three patients in a frontal plane, which are determined to be narrow (FIG. 2A), wide (FIG. 2B) and very wide (FIG. 2C) by using the facial morphologic index according to Garson (hereinafter—Garson index), while their visual inspection yields opposite results;

FIG. 3 shows a schematic block diagram of a method for performing a photometric cephalometric analysis according to one exemplary embodiment;

FIG. 4 explains how new cephalometric parameters used in the method of FIG. 3 are used to calculate a facial proportionality angle (FPA);

FIGS. 5A-5C show the faces of the same three patients in the frontal plane, demonstrating the FPAs calculated using the method of FIG. 3, compared to the Garson indices; and

FIGS. 6A-6D show faces of other four patients in the frontal plane, whose FPAs have been calculated using the method of FIG. 3.

### DETAILED DESCRIPTION OF THE INVENTION

Various embodiments of the present disclosure are further described in more detail with reference to the accompanying drawings. However, the present disclosure may be embodied in many other forms and should not be construed as limited to any certain structure or function discussed in the following description. In contrast, these embodiments are provided to make the description of the present disclosure detailed and complete.

According to the detailed description, it will be apparent to the ones skilled in the art that the scope of the present disclosure encompasses any embodiment thereof, which is disclosed herein, irrespective of whether this embodiment is implemented independently or in concert with any other embodiment of the present disclosure. For example, the method and apparatus disclosed herein may be implemented in practice by using any numbers of the embodiments provided herein. Furthermore, it should be understood that any embodiment of the present disclosure may be implemented using one or more of the elements presented in the appended claims.

The word “exemplary” is used herein in the meaning of “used as an illustration”. Unless otherwise stated, any embodiment described herein as “exemplary” should not be construed as preferable or having an advantage over other embodiments.

Any positioning terminology, such as “left”, “right”, “top”, “bottom”, “above” “below”, “upper”, “lower”, “horizontal”, “vertical”, etc., may be used herein for convenience to describe one element's or feature's relationship to one or more other elements or features in accordance with the figures. It should be apparent that the positioning terminology is intended to encompass different orientations of the apparatus disclosed herein, in addition to the orientation(s) depicted in the figures. As an example, if one imaginatively rotates the apparatus in the figures 90 degrees clockwise, elements or features described as “left” and “right” relative to other elements or features would then be oriented, respectively, “above” and “below” the other elements or features. Therefore, the positioning terminology used herein should not be construed as any limitation of the present disclosure.

When working with the face in various areas of aesthetic medicine, be it plastic reconstructive surgery, maxillofacial surgery or cosmetology, an important task is to harmonize its proportions. To assess the proportionality of the face, anthropometric measurements have long been proposed and carried out using cephalometric points and indices.

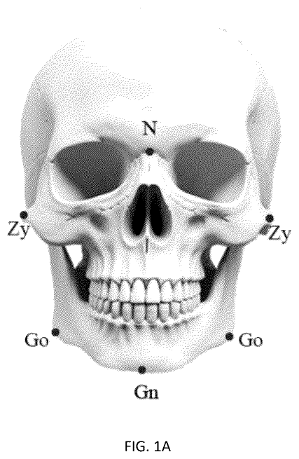

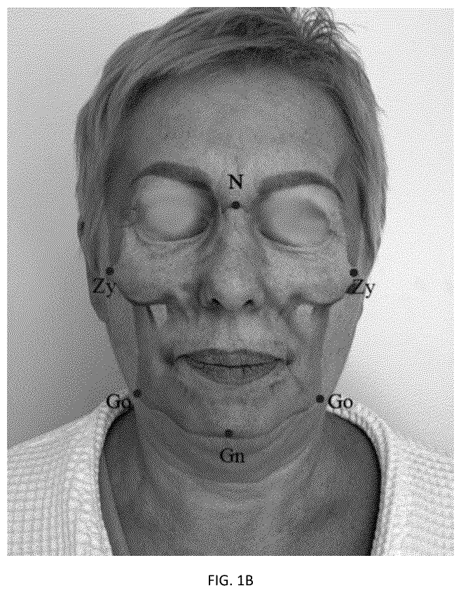

FIGS. 1A and 1B show the location of craniometric points on the skull (FIG. 1A) and relative to the soft tissue cover of the head (FIG. 1B), which are used to calculate the Garson index. This index is used to classify face shapes based on the relationship between facial height and width. It is calculated by dividing the morphological face height (measured from the nasion (n) to the gnathion (gn)) by the morphological face width or bizygomatic breadth (measured between the zygomatic arches (zy-zy)), then multiplying by 100. The resulting value is then categorized into different face types like hypereuryprosopic, euryprosopic, mesoprosopic, leptoprosopic, and hyperleptoprosopic (see Table 1 below).

TABLE 1
Dependence of the face shape on the Garson index

| Face shape | Index, % |
|---|---|
| Very wide (hypereuryprosopic) | 78.9 or less |
| Wide (euryprosopic) | 79.9-83.9 |
| Medium (mesoprosopic) | 84.0-87.9 |
| Narrow (leptoprosopic) | 88.0-92.2 |
| Very narrow (hyperleptoprosopic) | 93.0 or more |

However, if the Garson index is used to measure the proportions of the face, the indicators often do not correspond to the visual assessment of the face, and also require additional research methods in the form of teleroentgenograms or 3D radiograms, as well as measurements of the width and height of the face with pressing the soft tissues of the patient's face to the bones of the facial skull, which is inconvenient in the routine practice of a plastic surgeon or cosmetologist. On the contrary, the definition of soft tissue points, although convenient, again does not correspond one hundred percent to the visual assessment, often giving out opposite characteristics.

The present author conducted a clinical analysis of cephalometric parameters of 150 patients aged 37 to 76 years who had complaints of age-related changes in the oval of the face and neck. Each patient was examined clinically by performing cephalometric calculations of the morphological face height (i.e., n-gn) and the morphological face width by craniometry with compression of soft tissues and soft tissue points of the face using a caliper, calculating the facial morphologic indices according to Garson and Farkas, and entering the data into a map. Each patient was photographed at the preoperative stage and 1 year after surgery. The photo shooting was carried out from standard angles and with the positioning of the head along the Frankfurt horizontal plane. The photographs were compared according to a single angle and focal length to eliminate the effect of optical distortion on the proportions of the face. Then, based on the completed images, a photometric cephalometric analysis was also carried out with the measurement of the morphological face height and the inter-cheek distance and with the calculation of the indices according to Garson and Farkas.

The data obtained were compared and had similar numerical characteristics, but in most cases had contradictory values: some faces were narrow according to measurements, but on visual inspection they were considered wide, and vice versa.

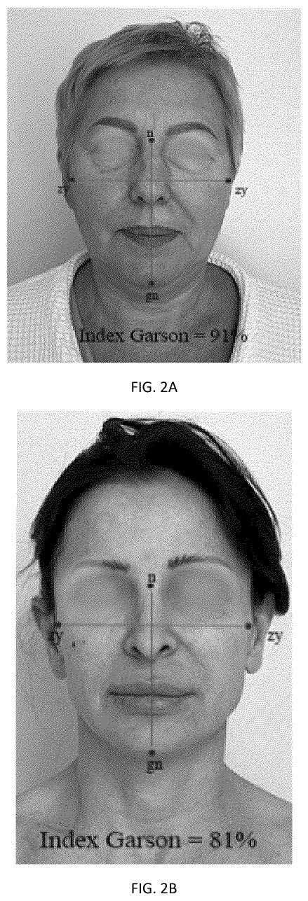

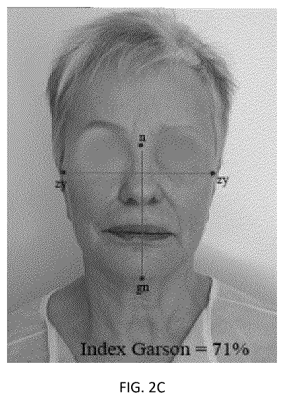

FIGS. 2A-2C show faces of three patients in a frontal plane, which are determined to be narrow (FIG. 2A), wide (FIG. 2B) and very wide (FIG. 2C) by using the Garson index, while their visual inspection yields opposite results. More specifically, the Garson index of the face shown in FIG. 2A is 91%, thereby characterizing the patient's face as narrow; upon its visual assessment the face appears average or medium. The Garson index of the face shown in FIG. 2B is 81%, thereby characterizing the patient's face as wide; upon its visual assessment the face appears average or medium. The Garson index of the face shown in FIG. 2C is 71%, thereby characterizing the patient's face as very wide; upon its visual assessment the face appears narrow.

The absurdity of the data obtained can be explained by the incorrect choice of the cephalometric parameters for evaluating the face. This is because the study of the distances between the bone cephalometric points does not give an accurate idea of the condition of the soft-tissue cover of the face as a whole and its deformation, which can develop during life. It has been proven that the thickness of soft tissues varies significantly at different levels of the face in people of different genders and ages. In addition, the different thickness of the soft-tissue cover of the face can vary significantly, for example, against the background of the use of various fillers, lipolytics, as well as with an increase or decrease in the weight of an individual.

Thus, the conventional measurements based on the Garson (or Farkas) index do not take into account age-related changes that affect the face height and width. In other words, these measurements do not reflect the true state of the volume, thickness and position of the soft tissues of the face. At the same time, it is known that the volume of soft tissues on the face can vary greatly depending on the individual's weight, ethnic characteristics, age, gender characteristics, teeth bite condition, cosmetology history, etc.

Given the above, some new cephalometric parameters are needed, with which it is possible to measure and characterize the proportions of the face taking into account the volume and thickness of soft tissues.

In orthognathic literature, it is common to describe soft tissue points that are projections of craniometric points with lowercase letters. To study the new cephalometric parameters, the present author has used the same soft-tissue points: 1) zy, located on the surface of the skin above the maximum point of the zygomatic bone on both sides; 2) the gnation point (gn), located at the point of maximum projection of the soft-tissue part of the chin in the sagittal plane. In addition, the present author has proposed the following new names:
1) The zygion (zy)-zygion (zy) distance, which in the available literature is also referred to as the distance between the bone cephalometric points Zy-Zy, namely the “interzygomatic distance”, which in the opinion of the present author confuses the measurements. Therefore, it has been suggested to leave the name “interzygomatic distance” behind the line between the bone points Zy-Zy and call the distance between the skin points zy-zy “visual interzygomatic distance”, characterizing the visual width of the face at the level of the zygomatic arches.
2) By analogy, the distance between the soft-tissue point nasion (n) and the soft-tissue chin point gnathion (gn) is proposed to be called the “visual morphological height of the face”, which allows one to take into account the elongation of the face in the case of age-related or iatrogenic ptosis of the soft tissues of the chin.

Due to the fact that the value of the visual interzygomatic distance and the Garson index do not give a reliable idea of the width of the face and its proportionality, and cephalometric clinical calculations are time-consuming, the present author has also proposed to calculate the angle between the points zy on both sides and the point gn, which is the vertex of the angle. This angle is called the Facial Proportionality Angle (FPA). The FPA is most convenient to measure from full-face photographs of the patient's face with the patient's head positioning along the Frankfurt horizontal plane. In this case, the scale of the photo does not matter, since the FPA is a relative parameter. According to the present author, this parameter allows one to accurately characterize the shape of the face in accordance with visual perception.

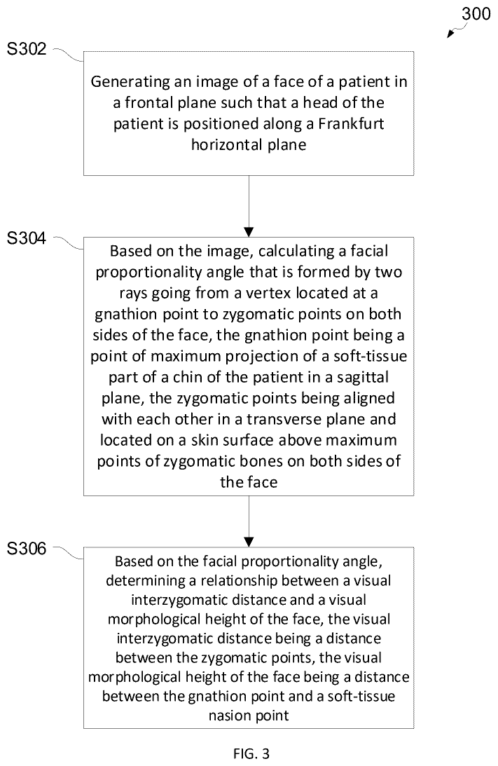

FIG. 3 shows a schematic block diagram of a method 300 for performing a photometric cephalometric analysis according to one exemplary embodiment. The method 300 starts with a step S302, in which an image of a face of a patient in a frontal plane is generated such that his/her head is positioned along the Frankfurt horizontal plane. For patient imaging, any camera may be used, provided its resolution and image quality are sufficient to clearly visualize and identify the key anatomical or craniometric points required to calculate the above-mentioned new cephalometric parameters.

Next, the method 300 proceeds to a step S304, in which the image is then used to calculate an FPA for the patient, with the FPA being formed by two rays going from a vertex located at a gnathion point to zygomatic points on both sides of the face. The gnathion point is a point of maximum projection of a soft-tissue part of the patient's chin in a sagittal plane, and the zygomatic points are aligned with each other in a transverse plane and located on a skin surface above maximum points of zygomatic bones on both sides of the face.

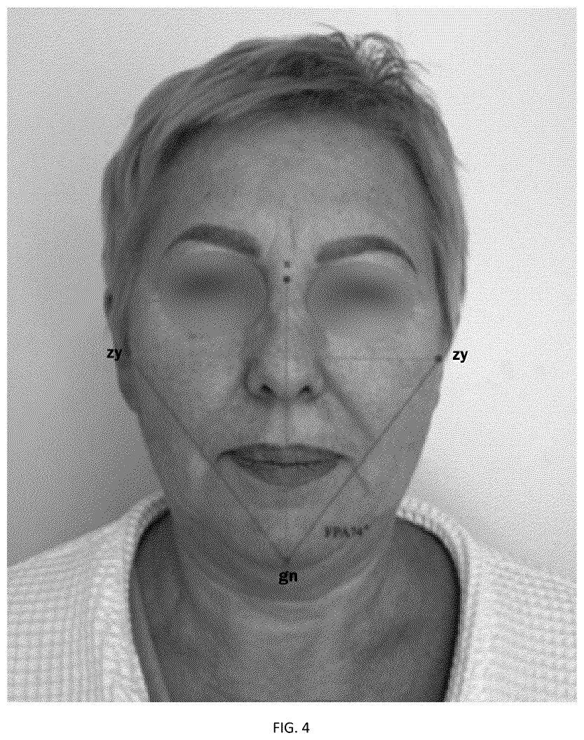

FIG. 4 explains how the new cephalometric parameters used in the method 300 are used to calculate the FPA. The points zy are located on the skin surface above the most protruding point of the zygomatic bone on both sides. The zy-zy distance, or visual interzygomatic distance, characterizes the true (visual) width of the face at the level of the zygomatic arches. The point gn located at the point of maximum projection of the soft-tissue part of the chin in the sagittal plane, and this point is extremely important for visual perception of the height of the face and assessment of its aesthetic parameters and proportions. The FPA is defined between the points zy on both sides and the point gn. This parameter characterizes the ratio of the visual width of the face to its morphological height.

Once the FPA is calculated, the method 300 goes on to a step S306, in which the calculated FPA is used to determine a relationship between the visual interzygomatic distance and the visual morphological height of the face. As noted earlier, the visual interzygomatic distance is a distance between the zygomatic points, and the visual morphological height of the face is a distance between the gnathion point and a soft-tissue nasion point.

It should be noted that the steps S304 and S306 can be performed by a processor included in a computer, which may be caused (when executing processor-executable instructions stored in a memory of the computer) to receive the image generated by the camera (e.g., directly from the camera or by downloading it from a remote or local storage of images of patients' faces) and process it to determine the FPA first and then the relationship between the visual interzygomatic distance and the visual morphological height of the patient's face. The processor may use a machine-learning (ML) model to perform at least one of the steps S304 and S306; in this case, the ML model may comprise a trained neural network configured to receive the image as input data, identify the points zy and gn in the image, plot the FPA based on the points zy and gn and calculate its value, determine the facial proportions (associated with the relationship between the zy-zy distance and the n-gn distance) by analyzing the calculated FPA, and then output the result of said analysis (i.e., whether the patient's face is very wide, wide, very narrow, narrow, or medium).

According to the data obtained by the present author, an FPA value greater than 80 degrees characterizes an excessively wide and short face, an FPA value from 75 to 79 degrees usually characterizes a short and wide face (i.e., a patient has equal values of the visual width and visual morphological height of the face), an FPA value from 70 to 74 degrees characterizes an average or medium face in height and width, and an FPA value of less than 70 degrees is typical for narrow and long faces. According to the present author's observations, faces with an FPA value in the range of 70-74 degrees look the most proportionally and harmoniously, they are characterized by the most aesthetic ratio of the visual width and visual height of the face.

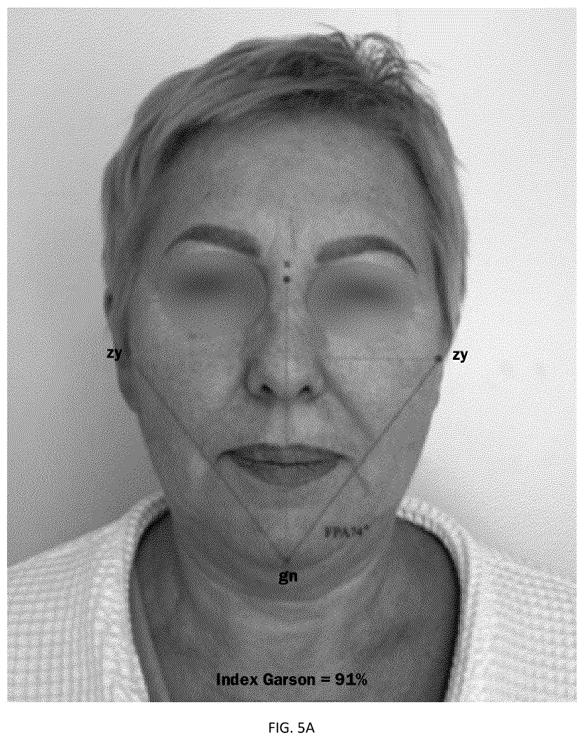

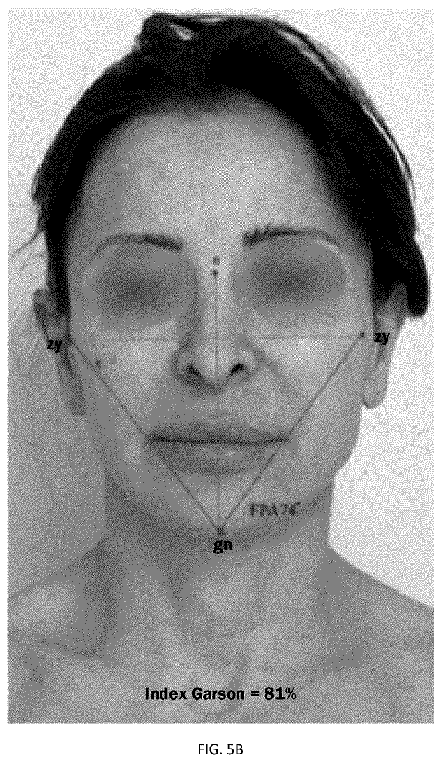

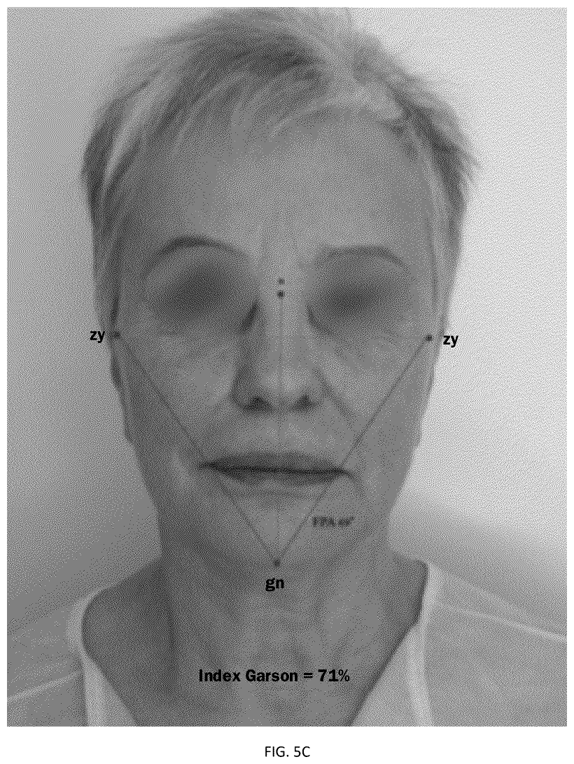

FIGS. 5A-5C show the faces of the same three patients in the frontal plane, demonstrating the FPAs calculated using the method 300, compared to the Garson indices.

In FIG. 5A, the FPA of 74 degrees characterizes the patient's face as average, the face does not look wide due to the large visual morphological height of the face. At the same time, the Garson index of 91% characterizes this face as narrow, which does not correspond to the visual assessment.

In FIG. 5B, the FPA of 74 degrees also characterizes the patient's face as average, due to the ratio of the visual morphological height and width of the face, and the face looks harmonious. However, the Garson index of 81% characterizes this face as wide, which does not correspond to the visual assessment.

In FIG. 5C, the FPA of 69 degrees characterizes the patient's face as narrow, due to the small morphological width of the face. However, the Garson index of 71% characterizes this face as very wide, which does not correspond to the visual assessment.

Thus, by measuring only the FPA, the researcher (e.g., physician) does not depend on the absolute values of the visual height of the face, he/she does not need to calculate the Garson index—it is enough to use a photograph of a patient of any size with the position of the patient's head oriented along the Frankfurt horizontal plane, a computer or protractor to determine the aesthetics and proportionality of the face, which greatly facilitates the application of these parameters in routine practice, allowing one to evaluate the aesthetics of the face even remotely, using the acquired knowledge to plan surgical or cosmetological facial correction.

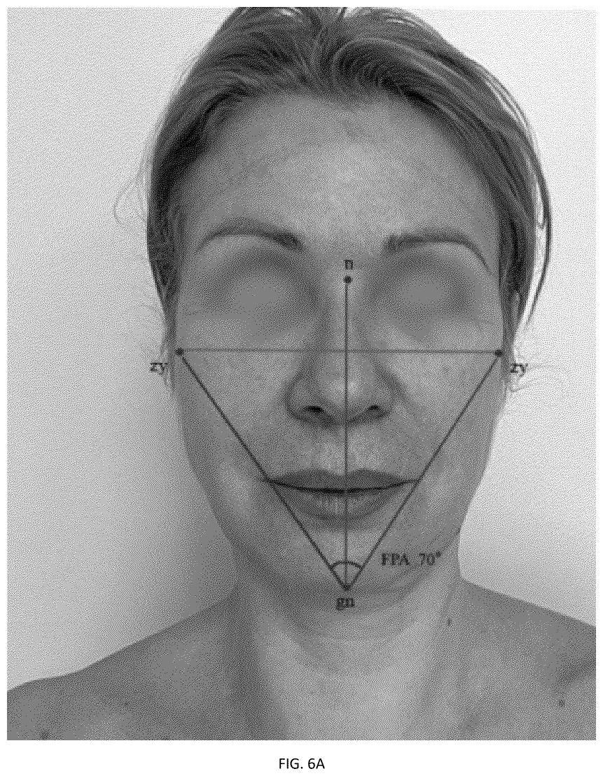

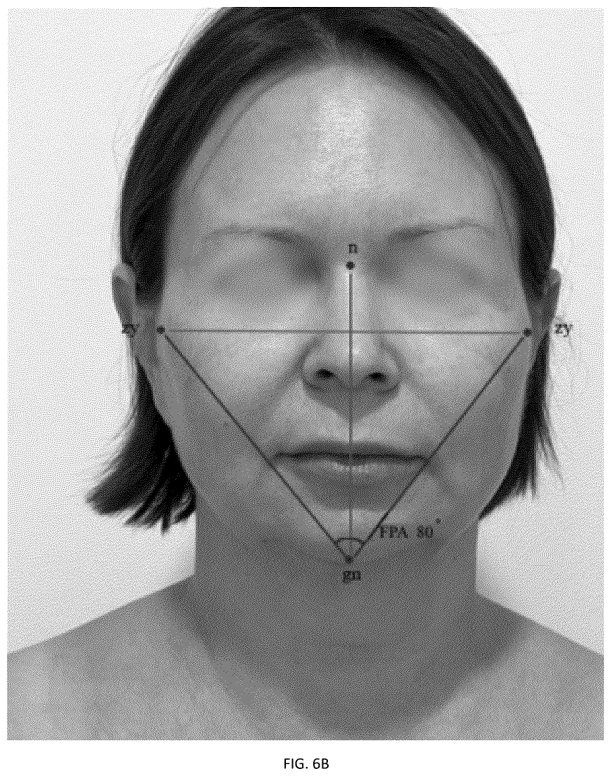

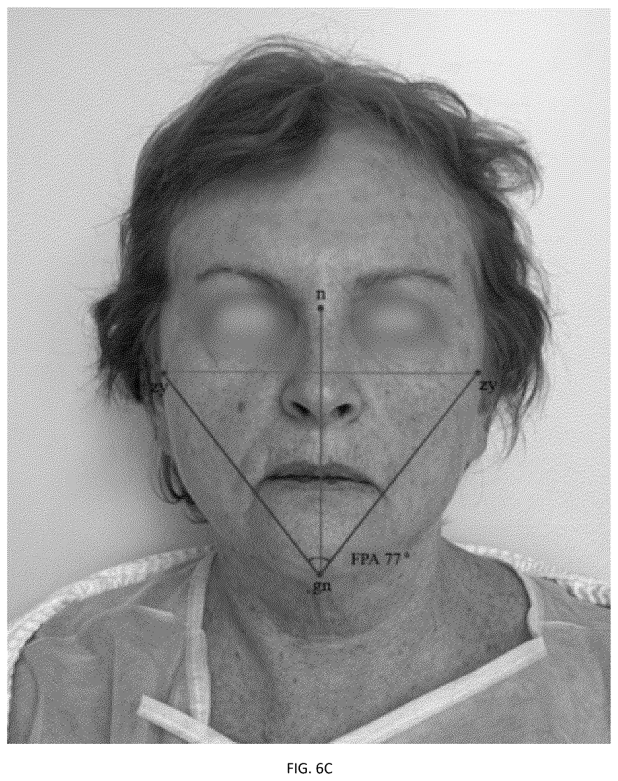

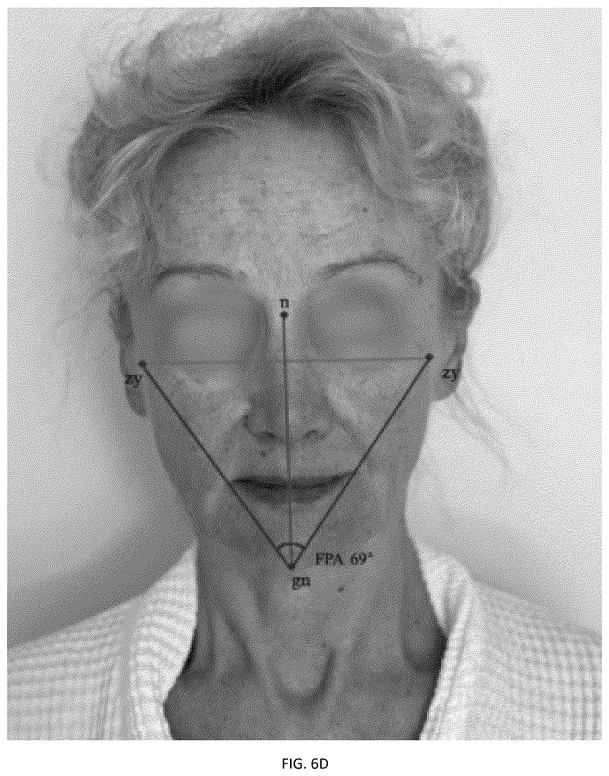

FIGS. 6A-6D show faces of other four patients in the frontal plane, whose FPAs have been calculated using the method of FIG. 3.

More specifically, FIG. 6A shows an example of a patient with an FPA value of 70 degrees. As noted earlier, faces with such FPA values look proportional and harmonious, they are characterized by the most aesthetic ratio of the visual width and visual height of the face.

In FIG. 6B, a person with an FPA value of 80 degrees is shown. Persons with FPA values of 80 degrees or more have a wide visual interzygomatic distance, prevailing over the visual morphological height of the face. The large size of the visual interzygomatic (zy-zy) distance is explained either by the pronounced size of the zygomatic arches themselves, or by the excessive thickness of subcutaneous soft tissues in their lateral sections. Another reason for the disproportionality of these faces is the insufficient visual height of the face associated with a deficiency in chin projection for various reasons.

FIG. 6C shows an example of a patient with an FPA value of 77 degrees. Patients with an FPA value of 75-79 degrees are characterized by the visual interzygomatic (zy-zy) distance almost equal to the visual morphological height (n-gn) of the face, which is why the face looks quite voluminous, without any aesthetic accents and has a circular shape. This is usually due to a fairly large volume of soft tissue in the zygomatic and lateral parts of the face.

In FIG. 6D, a patient with an FPA value of 69 degrees is shown. Patients having an FPA value of less than 70 degrees are characterized by the visual interzygomatic (zy-zy) distance significantly less than the visual morphological height (n-gn) of the face, usually due to the lack of expression of the zygomatic arches or insufficient thickness of the soft tissues in the zygomatic areas. Another reason for the “elongation of the face” may be the excessive projection of the gnathion point, due to the posturing of the soft tissues of the chin or orthognathic deformation.

Although the exemplary embodiments of the present disclosure are described herein, it should be noted that any various changes and modifications could be made in the embodiments of the present disclosure, without departing from the scope of legal protection which is defined by the appended claims. In the appended claims, the word “comprising” does not exclude other elements or operations, and the indefinite article “a” or “an” does not exclude a plurality. The mere fact that certain measures are recited in mutually different dependent claims does not indicate that a combination of these measures may not be used to advantage.

1. A method for performing a photometric cephalometric analysis, comprising:
generating an image of a face of a patient in a frontal plane such that a head of the patient is positioned along a Frankfurt horizontal plane;
based on the image, calculating a facial proportionality angle, the facial proportionality angle being formed by two rays going from a vertex located at a gnathion point to zygomatic points on both sides of the face, the gnathion point being a point of maximum projection of a soft-tissue part of a chin of the patient in a sagittal plane, the zygomatic points being aligned with each other in a transverse plane and located on a skin surface above most protruding points of zygomatic bones on both sides of the face; and
based on the facial proportionality angle, determining a relationship between a visual interzygomatic distance and a visual morphological height of the face, the visual interzygomatic distance being a distance between the zygomatic points, the visual morphological height of the face being a distance between the gnathion point and a soft-tissue nasion point.

2. The method of claim 1, wherein said determining comprises:
if the facial proportionality angle is less than 70 degrees, determining that the visual morphological height of the face prevails over the visual interzygomatic distance, meaning that the face of the patient is long and narrow; and
if the facial proportionality angle is more than 75 degrees, determining that the visual interzygomatic distance prevails over the visual morphological height of the face, meaning that the face of the patient is short and wide.

3. An apparatus for performing a photometric cephalometric analysis, comprising:
at least one processor; and
at least one memory coupled to the at least one processor and storing processor-executable instructions,
wherein the processor-executable instructions are configured, when executed by the at least one processor, to cause the at least one processor to:
receive an image of a face of a patient in a frontal plane, the image showing a head of the patient positioned along a Frankfurt horizontal plane;
based on the image, calculate a facial proportionality angle, the facial proportionality angle being formed by two rays going from a vertex located at a gnathion point to zygomatic points on both sides of the face, the gnathion point being a point of maximum projection of a soft-tissue part of a chin of the patient in a sagittal plane, the zygomatic points being aligned with each other in a transverse plane and located on a skin surface above most protruding points of zygomatic bones on both sides of the face;
based on the facial proportionality angle, determine a relationship between a visual interzygomatic distance and a visual morphological height of the face, the visual interzygomatic distance being a distance between the zygomatic points, the visual morphological height of the face being a distance between the gnathion point and a soft-tissue nasion point; and
output, to a user, the determined relationship between the visual interzygomatic distance and the visual morphological height of the face.

4. The apparatus of claim 3, wherein the at least one processor is caused to:
if the facial proportionality angle is less than 70 degrees, determine that the visual morphological height of the face prevails over the visual interzygomatic distance, meaning that the face of the patient is long and narrow; and
if the facial proportionality angle is more than 75 degrees, determining that the visual interzygomatic distance prevails over the visual morphological height of the face, meaning that the face of the patient is short and wide.
